(19) United States
(12) Patent Application Publication
(10) Pub. No.: US 2024/0390457 A1
(43) Pub. Date: Nov. 28, 2024

(54) COMPOSITION AND METHOD OF TREATING BRAIN CANCER

(71) Applicant: Morehouse School of Medicine, Atlanta, GA (US)

(72) Inventors: Zhigang XIONG, Atlanta, GA (US); Tiandong LENG, Snellville, GA (US)

(21) Appl. No.: 18/324,461

(22) Filed: May 26, 2023

(51) Int. Cl. A61K 38/17; A61K 31/4965; A61P 35/00

(52) U.S. Cl. CPC A61K 38/1767; A61K 31/4965; A61P 35/00

(57) ABSTRACT

The present application discloses a method of treating brain cancer, comprising administering a therapeutically effective amount of an acid sensing ion channel 1a (ASIC1a) inhibitor to an tumorigenic subject in order to reduce injury resulting from brain cancer. Also disclosed are compositions for treatment of brain cancer, comprising: an acid sensing ion channel 1a (ASIC1a) inhibitor disposed in a vehicle at a concentration that provides a therapeutically effective amount of the ASIC1a inhibitor for treatment of brain cancer when administered to an tumorigenic subject. A method of manufacturing a medicament for treatment of brain cancer is also disclosed.

This application is supported by certain government grants. The government has certain rights in this application.

### FIELD

The present application generally relates to methods and compositions for treatment of brain cancer, which may include approaches for reducing injury resulting from brain cancer and/or for identifying drugs for brain cancer treatment, in particular, by selectively inhibiting one or more members of the acid sensing ion channel (ASIC) family.

### BACKGROUND

Glioblastoma is a broad category of tumors derived from glial cells that support nerve cells in the brain and spinal cord. Every year, about 100,000 people worldwide are diagnosed with glioblastoma multiforme, a malignant glioma. Although it represents only one to two percent of all newly diagnosed cancers, glioblastoma is one of the most disabling and lethal diseases. The treatment and prognosis of glioblastoma depend on a series of factors, such as age, tumor type, and location of the tumor in the brain. The recurrence and poor prognosis of malignant glioma are mainly due to its strong ability of uncontrolled proliferation and aggressive invasion. The infiltrative growth makes surgical removal very difficult, sometimes impossible. In addition, the high resistance of malignant glioma cells to therapies and the vulnerability of the central nervous system hamper the antitumor interventions. The effective therapies, are needed to prolong the survival of glioblastoma patients.

Acid-sensing ion channels (ASICs) are voltage-insensitive cationic channels activated by extracellular acidification. ASICs form a subfamily of the epithelial sodium channels/degenerin (ENaC/Deg) family. Four different genes encode six ASIC subunits: ASIC1a, ASIC1b, ASIC2a, ASIC2b, ASIC3 and ASIC4. They can form functional hetero/homotrimeric channels that are activated by acidic pH.

### SUMMARY

Aspects of the application provide a system, including methods and compositions, for treatment of brain cancer.

An aspect of the application is a method of treating brain cancer, comprising: administering a therapeutically effective amount of an acid sensing ion channel 1a (ASIC1a) inhibitor to a tumorigenic subject in order to reduce injury resulting from brain cancer.

An aspect of the application is a method of treating brain cancer, comprising: administering a therapeutically effective amount of a cystine knot peptide to a tumorigenic subject in order to reduce injury resulting from brain cancer.

An aspect of the application is a composition for treatment of brain cancer, comprising: an acid sensing ion channel 1a (ASIC1a) inhibitor disposed in a vehicle at a concentration that provides a therapeutically effective amount of the ASIC1a inhibitor for treatment of brain cancer when administered to an tumorigenic subject. In certain embodiments, in addition to ASIC1a inhibitors such as amiloride and PcTX1, the composition and methods herein may make use of ASIC-siRNA.

An aspect of the application is a method of manufacturing a medicament for treatment of brain cancer, comprising: obtaining an acid sensing ion channel 1a (ASIC1a) inhibitor; and combining the ASIC1a inhibitor with a vehicle to produce a medicament having a therapeutically effective concentration of the inhibitor for administration to an tumorigenic subject for treatment of brain cancer.

An aspect of the application is a use of an acid sensing ion channel 1a (ASIC1a) inhibitor for the manufacture of a medicament to treat brain cancer.

An aspect of the application is a method of manufacturing a medicament for treating brain cancer, comprising formulating an ASIC1a inhibitor into such a medicament.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows expression of ASIC1 and electrophysiological characteristics of ASICs currents in glioma A172 and U87MG cells. (A) Representative Western blot bands showing ASIC1 proteins in A172 and U87MG cells. (B) Representative traces showing the ASIC current in A172 and U87MG cells. (C) Representative traces and (D) statistical results showing the inhibitory effect of PcTX1 (20 nM) on ASIC current in U87MG cells. Data are expressed as mean±SEM. n=5. ***P<0.001 by Students' t-test.

FIG. 2 shows effects of acidosis and PcTX1 on proliferation of A172 and U87MG cells. (A) Representative micrographs of 24 h and 72 h and (B) statistical results of 24 h, 48 h and 72 h showing the presence or lack of effects of extracellular weak acid pH 7.0 and PcTX1 on the proliferation of A172 cells. (C) Representative micrographs of 24 h and 72 h and (D) statistical results of 24 h, 48 h and 72 h showing the presence or lack of effect of extracellular weak acid and PcTX1 on the proliferation of U87MG cells. Data are expressed as mean±SEM. n=3. Scale bar=100 μm. One-way ANOVA followed by Tukey's multiple comparison test.

FIG. 3 shows effects of extracellular acidosis and PcTX1 on migration of A172 and U87MG cells by wound healing assay. (A) Representative micrographs and (B) statistical results showing the effect of extracellular weak acid and PcTX1 on migration of A172 cells by wound-healing assay. (C) Representative micrographs and (D) statistical results showing the effect of extracellular acid and PcTX1 on the migration of U87MG cells by wound-healing assay. Data are expressed as mean±SEM. n=3-4. Scale bar=250 μm. *P<0.05, **P<0.01, by one-way ANOVA followed by Tukey's multiple comparison test.

FIG. 4 shows effects of extracellular acidosis and PcTX1 on migration of A172 and U87MG cells by transwell. (A) Representative micrographs and (B) statistical results showing the effects of extracellular acid and PcTX1 on the migration of A172 cells by transwell assay. (C) Representative micrographs and (D) statistical results showing the effects of extracellular acid and PcTX1 on the migration of U87MG cells by transwell assay. Data are expressed as mean±SEM. n=4-5. Scale bar=100 μm. *P<0.05, by one-way ANOVA followed by Tukey's multiple comparison test.

FIG. 5 shows silencing ASIC1 inhibits the migration of U87MG cells. (A) Representative Western blot bands and (B) quantification of the ASIC1 expression showing the transfection efficiency of siRNA-ASIC1 in U87MG cells. (C) Representative micrographs and (D) statistical results showing that knockdown of ASIC1 has no obvious effect on the proliferation of U87MG cells both at 24 h and 72 h without or with the treatment of extracellular weak acid. (E) Representative micrographs and (F) statistical results showing the effect of siRNA-Control or siRNA-ASIC1 on acid-induced migration of U87MG cells by wound-healing assay. (G) Representative micrographs and (H) statistical results showing the effects of siRNA-Control or siRNA-ASIC1 on acid-induced migration of U87MG cells by transwell assay. Data are expressed as mean±SEM. n=3-5. Black bar=250 μm. White bar=100 μm. *P<0.05, **P<0.01, ***P<0.001, by one-way ANOVA followed by Tukey's multiple comparison test.

### DETAILED DESCRIPTION

Herein incorporated by reference is the sequence listing filed with the USPTO named as “1013-712.xml” which was created on May 26, 2023, and the size of the XML file is 7,024 in bytes.

Unless defined otherwise, all technical and scientific terms used herein have the same meanings as commonly understood by one of ordinary skill in the art to which this application belongs. All publications and patents specifically mentioned herein are incorporated by reference in their entirety for all purposes including describing and disclosing the chemicals, instruments, statistical analyses, and methodologies which are reported in the publications which might be used in connection with the application. All references cited in this specification are to be taken as indicative of the level of skill in the art. Nothing herein is to be construed as an admission that the application is not entitled to antedate such disclosure by virtue of prior invention.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

It must be noted that as used herein and in the appended claims, the singular forms “a,” “an,” and “the” include plural reference unless the context clearly dictates otherwise. Further, the terms “a” (or “an”), “one or more” and “at least one” can be used interchangeably herein. It is also to be noted that the terms “comprising,” “including,” “characterized by” and “having” can be used interchangeably. Further, any reactant concentrations described herein should be considered as being described on a weight to weight (w/w) basis, unless otherwise specified to the contrary (e.g., mole to mole, weight to volume (w/v), etc.).

In the specification and in the claims, the terms “including” and “comprising” are open-ended terms and should be interpreted to mean “including, but not limited to . . . ” These terms encompass the more restrictive terms “consisting essentially of” and “consisting of.”

As used in this specification and the appended claims, the singular forms “a,” “an” and “the” include plural referents unless the content clearly dictates otherwise.

The term “ASIC1a inhibitor composition” and “pharmaceutical composition” are used herein with reference to a composition comprising ASIC1a inhibitor and at least one pharmaceutically acceptable carrier. When referring to these compositions with regard to dosages, the weights are given in terms of the amount by weight of ASIC1a inhibitor.

The term “subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject suspected of having” means a subject exhibiting one or more clinical indicators of a microbial disease or condition.

A “subject in need thereof” means a subject identified as in need of a therapy or treatment.

A “therapeutic effect” relieves, to some extent, one or more of the symptoms of a microbial disease or disorder. “Curing” means that the symptoms of active disease are eliminated. However, certain long-term or permanent effects of the disease may exist even after a cure is obtained (such as tissue damage and the like).

The phrase “therapeutically effective amount” as used herein refers to an amount of ASIC1a inhibitor that ameliorates, attenuates, or eliminates one or more of the symptoms of a particular disease or condition or prevents, modifies, or delays the onset of one or more of the symptoms of a microbial disease or condition.

“Treat”, “treatment,” and “treating,” as used herein, refer to administering an ASIC1a inhibitor composition for prophylactic and/or therapeutic purposes. The term “prophylactic treatment” refers to treating a patient who does not yet have the relevant disease or disorder, but who is susceptible to, or otherwise at risk of, a particular disease or disorder, whereby the treatment reduces the likelihood that the patient will develop the disease or disorder. The term “therapeutic treatment” refers to administering treatment to a patient already having a disease or disorder.

“Preventing” or “prevention” refers to delaying or forestalling the onset, development or progression of a microbial condition or disease for a period, including weeks, months, or years.

“Amelioration” means a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“Administering” means providing a pharmaceutical agent or composition to a subject, and includes, but is not limited to, administering by a medical professional and self-administering.

Administration of the ASIC1a inhibitor compositions of the present application can be via any of the accepted modes of administration for agents that serve similar utilities including, but not limited to, orally, subcutaneously, intravenously, intranasally, topically, transdermally, intraperitoneally, intramuscularly, intrapulmonarily, vaginally, rectally, or intraocularly.

“Parenteral administration,” means administration through injection or infusion. Parenteral administration includes, but is not limited to, subcutaneous administration, intravenous administration, intramuscular administration, intraarterial administration, and intracranial administration.

“Subcutaneous administration” means administration just below the skin.

“Intravenous administration” means administration into a vein.

“Intraarterial administration” means administration into an artery.

“Pharmaceutical composition” means a mixture of substances suitable for administering to an individual that includes a pharmaceutical agent. For example, a pharmaceutical composition may comprise a modified oligonucleotide and a sterile aqueous solution.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable carrier” or “pharmaceutically acceptable excipient” includes all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form, however, does not imply that the dosage form is administered once per day or once per course of therapy.

Weak acid (e.g., pH 7.0) promotes the migration of malignant glioma cells, at least in part, by activating ASIC1 channels.

Glioblastoma is the most aggressive and lethal tumor in the central nervous system in adult and has poor prognosis due to strong proliferation and aggressive invasion capacity. Acidic microenvironment is commonly observed in tumor tissues but the exact role of acidosis in the pathophysiology of glioblastoma and underlying mechanisms remain unclear. Acid-sensing ion channels (ASICs) are proton-gated cation channels activated by low extracellular pH. ASICs are involved in the pathogenesis of some tumors, such as lung cancer and breast cancer. The compositions and methods described herein can be used in the context of all cancers having acidic local environment so ASICs are supposed to be activated.

Other cancers for which the compositions and methods described herein may be used include prostate cancer, ovarian cancer, renal cancer, cervical cancer, bladder cancer, testicular cancer, leukemia, Kaposi sarcoma, myeloma, melanoma, and kidney cancer.

The present teachings provide a system, including methods and compositions, for treatment of brain cancer. The methods may include approaches for reducing injury resulting from brain cancer and/or for identifying drugs for brain cancer treatment. The methods selectively may inhibit one or more members of the acid sensing ion channel (ASIC) family, to provide a targeted therapy for brain cancer treatment.

This study investigated the expression of ASIC1 in human glioma cell lines (U87MG and A172) and its effect on the proliferation and migration of these cells. The results demonstrated that ASIC1 is functionally expressed in U87MG and A172 cells. Treatment with extracellular weak acid (pH 7.0) has no effect on the proliferation but increases the migration of the two cell lines. Application of PcTX1, a specific inhibitor of ASIC1a and ASIC1a/2b channels, or knocking down ASIC1 by siRNA, can abolish the effect of weak acid-induced cell migration. These results indicate that ASIC1 mediates extracellular weak acid induced migration of human malignant glioma cells and can serve as a therapeutic target for malignant glioma in human.

### Method of Treating Brain Cancer Using Amiloride Derivatives

An aspect of the application is a method of treating brain cancer, comprising: administering a therapeutically effective amount of an acid sensing ion channel 1a (ASIC1a) inhibitor to a tumorigenic subject in order to reduce injury resulting from brain cancer. In certain embodiments, the ASIC1a inhibitor is an amiloride derivative (see, e.g., Leng et al., CNS Neurosci Ther 2016 June; 22 (6): 468-76, which is incorporated herein by reference). In certain embodiments, the amiloride derivative is benzamil.

The system of the present teachings may provide treatment of tumorigenic subjects to reduce tumorigenic injury to the subjects. An tumorigenic subject, as used herein, is any person (a human subject) or animal (an animal subject) that has brain cancer, an brain cancer-related condition, a history of brain cancer, and/or a significant chance of developing brain cancer after treatment begins and during a time period in which the treatment is still effective.

The tumorigenic subject may be an animal. The term “animal,” as used herein, refers to any animal that is not human. Exemplary animals that may be suitable include any animal with a bloodstream, such as rodents (mice, rats, etc.), dogs, cats, birds, sheep, goats, non-human primates, etc. The animal may be treated for its own sake, e.g., for veterinary purposes (such as treatment of a pet). Alternatively, the animal may provide an animal model of brain cancer, to facilitate testing drug candidates for human use, such as to determine the candidates' potency, window of effectiveness, side effects, etc.

A brain cancer-related condition may be any consequence of brain cancer. The consequence may be substantially concurrent with the onset brain cancer (e.g., a direct effect of the brain cancer) and/or may occur substantially after brain cancer onset and/or even after the brain cancer is over (e.g., an indirect, downstream effect of the brain cancer, such reperfusion of tissue when brain cancer ends). Exemplary brain cancer-related conditions may include any combination of the symptoms (and/or conditions) listed above in Section I. Alternatively, or in addition, the symptoms may include local and/or systemic acidosis (pH decrease), hypoxia (oxygen decrease), free radical generation, and/or the like.

Tumorigenic subjects for treatment may be selected by any suitable criteria. Exemplary criteria may include any detectable symptoms of brain cancer, a history of brain cancer, an event that increases the risk of (or induces) brain cancer (such as a surgical procedure, trauma, administration of a medication, etc.), and/or the like. A history of brain cancer may involve one or more prior tumorigenic episodes. In some examples, a subject selected for treatment may have had an onset of brain cancer that occurred at least about one, two, or three hours before treatment begins, or a plurality of tumorigenic episodes (such as transient tumorigenic attacks) that occurred less than about one day, twelve hours, or six hours prior to initiation of treatment.

### Method of Treating Brain Cancer Using Other ASIC1a Inhibitors.

Another aspect of the application is a method of treating brain cancer, comprising: administering a therapeutically effective amount of a cystine knot peptide to an tumorigenic subject in order to reduce injury resulting from brain cancer.

### ASIC Inhibitors

Inhibitors of ASIC family members, as used herein, are substances that reduce (partially, substantially, or completely block) the activity or one or more members of the ASIC family, that is, ASIC1a, ASIC1b, ASIC2a, ASIC2b, ASIC3, and ASIC4, among others. In some examples, the inhibitors may reduce the channel activity of one or more members, such as the ability of the members to flux ions (e.g., sodium, calcium, and/or potassium ions, among others) through cell membranes (into and/or out of cells). The substances may be compounds (small molecules of less than about 10 kDa, peptides, nucleic acids, lipids, etc.), complexes of two or more compounds, and/or mixtures, among others. Furthermore, the substances may inhibit ASIC family members by any suitable mechanism including competitive, noncompetitive, uncompetitive, and/or mixed inhibition, among others.

The inhibitor may be an ASIC1a inhibitor that inhibits acid sensing ion channel 1a (ASIC1a). ASIC1a, as used herein, refers to an ASIC1a protein or channel from any species. For example, an exemplary human ASIC1a protein/channel is described in Waldmann, R., et al. 1997, Nature 386, pp. 173-177, which is incorporated herein by reference.

The expression “ASIC1a inhibitor” may refer to a product which, within the scope of sound pharmacological judgment, is potentially or actually pharmaceutically useful as an inhibitor of ASIC1a, and includes reference to substances which comprise a pharmaceutically active species and are described, promoted, or authorized as an ASIC1a inhibitor.

An ASIC1a inhibitor may be selective within the ASIC family. Selective inhibition of ASIC1a, as used herein, is inhibition that is substantially stronger on ASIC1a than on another ASIC family member(s) when compared (for example, in cultured cells) after exposure of each to the same (sub-maximal) concentration(s) of an inhibitor. The inhibitor may inhibit ASIC1a selectively relative to at least one other ASIC family member (ASIC1b, ASIC2a, ASIC2b, ASIC3, ASIC 4, etc.) and/or selectively relative to every other ASIC family member. The strength of inhibition for a selective inhibitor may be described by an inhibitor concentration at which inhibition occurs (e.g., an IC₅₀ (inhibitor concentration that produces 50% of maximal inhibition) or a Kᵢ value (inhibition constant or dissociation constant)) relative to different ASIC family members. An ASIC1a-selective inhibitor may inhibit ASIC1a activity at a concentration that is at least about two-, four-, or ten-fold lower (one-half, one-fourth, or one-tenth the concentration or lower) than for inhibition of at least one other or of every other ASIC family member. Accordingly, an ASIC1a-selective inhibitor may have an IC₅₀ and/or Kᵢ for ASIC1a inhibition that is at least about two-, four-, or ten-fold lower (one-half, one-fourth, or one-tenth or less) than for inhibition of at least one other ASIC family member and/or for inhibition of every other ASIC family member.

An ASIC1a-selective inhibitor, in addition to being selective, also may be specific for ASIC1a. ASIC1a-specific inhibition, as used herein, is inhibition that is substantially exclusive to ASIC1a relative to every other ASIC family member. An ASIC1a-specific inhibitor may inhibit ASIC1a at an inhibitor concentration that is at least about twenty-fold lower (5% of the concentration or less) than for inhibition of every other ASIC family member. Accordingly, an ASIC1a-specific inhibitor may have an IC₅₀ and/or Kᵢ for ASIC1a relative to every other member of the ASIC family that is at least about twenty-fold lower (five percent or less), such that, for example, inhibition of other ASIC family members is at least substantially (or completely) undetectable.

Any suitable ASIC inhibitor or combination of inhibitors may be used. For example, a subject may be treated with an ASIC1a-selective inhibitor and a nonselective ASIC inhibitor, or with an ASIC1a-selective inhibitor and an inhibitor to a non-ASIC channel protein, such as a non-ASIC calcium channel. In some examples, a subject may be treated with an ASIC1a-selective inhibitor and an inhibitor of NMDA receptors, such as a glutamate antagonist.

The inhibitor may be or include amiloride or amiloride derivatives.

The inhibitor may be or include a peptide. The peptide may have any suitable number of amino acid subunits, generally at least about ten and less than about one-thousand subunits. In some examples, the peptide may have a cystine knot motif. A cystine knot, as used herein, generally comprises an arrangement of six or more cysteines. A peptide with these cysteines may create a “knot” including (1) a ring formed by two disulfide bonds and their connecting backbone segments, and (2) a third disulfide bond that threads through the ring. In some examples, the peptide may be a conotoxin from an arachnid and/or cone snail species. For example, the peptide may be PcTx1 (psalmotoxin 1, see below), a toxin from a tarantula (Psalmopoeus cambridgei (Pc)).

| VARIANT | IDENTIFIER | POSITION |
|---|---|---|
| FULL LENGTH | (SEQ ID: 1) | EDCIPKWKGCVNRHGDCCEG |
| PcTx1 | | LECWKRRRSFEVCVPKTPKT |
| N-TERMINAL DEL. | (SEQ ID: 2) | CIPKWKGCVNRHGDCCEGLE |
| | | CWKRRRSFEVCVPKTPKT |
| C-TERMINAL DEL. | (SEQ ID: 3) | EDCIPKWKGCVNRHGDCCEG |
| | | LECWKRRRSFEVCVPKT |
| FULL C-TERMINAL | (SEQ ID: 4) | EDCIPKWKGCVNRHGDCCEG |
| DEL. | | LECWKRRRSFEVC |
| N & C-TERMINAL | (SEQ ID: 5) | CIPKWKGCVNRHGDCCEGLE |
| DEL. | | CWKRRRSFEVC |

In some examples, the peptide may be structurally related to PcTx1, such that the peptide and PcTx1 differ by at least one deletion, insertion, and/or substitution of one or more amino acids. For example, the peptide may have at least about 75%, at least about 80%, at least about 85%, at least about 90%, at least about 95%, at least about 96%, at least about 97%, and/or at least about 98% sequence identity with PcTx1 (see below). Further aspects of peptides that may be suitable as inhibitors are described herein in below Table 6.

| IDENTIFIER | POSITION |
|---|---|
| (SEQ ID: 1) | EDCIPKWKGCVNRHGDCCEG |
| | LECWKRRRSFEVCVPKTPKT |

Methods of alignment of amino acid sequences for comparison and generation of identity and similarity scores are well known in the art. Exemplary alignment methods that may be suitable include (Best Fit) of Smith and Waterman, a homology alignment algorithm (GAP) of Needleman and Wunsch, a similarity method (Tfasta and Fasta) of Pearson and Lipman, and/or the like. Computer algorithms of these and other approaches that may be suitable include, but are not limited to: CLUSTAL, GAP, BESTFIT, BLASTP, FASTA, and TFASTA.

As used herein, “sequence identity” or “identity” in the context of two peptides relates to the percentage of residues in the corresponding peptide sequences that are the same when aligned for maximum correspondence. In some examples, peptide residue positions that are not identical may differ by conservative amino acid substitutions, where amino acid residues are substituted for other amino acid residues with similar chemical properties (e.g. charge or hydrophobicity) and therefore are expected to produce a smaller (or no) effect on the functional properties of the molecule. Where sequences differ in conservative substitutions, the percent sequence identity may be adjusted upwards, to give a “similarity” of the sequences, which corrects for the conservative nature of the substitutions. For example, each conservative substitution may be scored as a partial rather than a full mismatch, thereby correcting the percentage sequence identity to provide a similarity score. The scoring of conservative substitutions to obtain similarity scores is well known in the art and may be calculated by any suitable approach, for example, according to the algorithm of Meyers and Miller, Computer Applic. Biol Sci., 4:11-17 (1988), e.g., as implemented in the program PC/GENE (Intelligenetics, Mountain View, Calif., USA).

Additional ASIC inhibitors may be identified for use as drugs to treat brain cancer. Identification may include (A) obtaining one or more ASIC inhibitors, and (B) testing the ASIC inhibitors on tumorigenic subjects.

One or more ASIC inhibitors, particularly ASIC1a inhibitors as described above, may be obtained. The inhibitors may be obtained by any suitable approach, such by screening a set of candidate inhibitors (e.g., a library of two or more compounds) and/or by rationale design, among others.

Screening may involve any suitable assay system that measures interaction between ASIC proteins and the set of candidate inhibitors. Exemplary assay systems may include assays performed biochemically (e.g., binding assays), with cells grown in culture (“cultured cells”), and/or with organisms, among others.

A cell-based assay system may measure an effect, if any, of each candidate inhibitor on ion flux in the cells, generally acid-sensitive ion flux. In some examples, the ion flux may be a flux of calcium and/or sodium, among others. The assay system may use cells expressing an ASIC family member, such as ASIC1a, or two or more distinct sets of cells expressing two or more distinct ASIC family members, such as ASIC1a and another ASIC family member(s), to determine the selectivity of each inhibitor for these family members. The cells may express each ASIC family member endogenously or through introduction of foreign nucleic acid. In some examples, the assay system may measure ion flux electrophysiologically (such as by patch clamp), using an ion-sensitive or membrane potential-sensitive dye (e.g., a calcium sensitive dye such as Fura-2), or via a gene-based reporter system that is sensitive to changes in membrane potential and/or intracellular ion (e.g., calcium) concentrations, among others. The assay system may be used to test candidate inhibitors for selective and/or specific inhibition of ASIC family members, particularly ASIC1a.

One or more ASIC inhibitors may be administered to an tumorigenic subject(s) to test the efficacy of the inhibitors for treatment of brain cancer. The tumorigenic subjects may be people or animals. In some examples, the tumorigenic subjects may provide an animal model system of brain cancer and/or tumor. Exemplary animal model systems include rodents (mice and/or rats, among others) with brain cancer induced experimentally. The brain cancer may be induced mechanically (e.g., surgically) and/or by administration of a drug, among others. In some examples, the brain cancer may be induced by occlusion of a blood vessel, such as by constriction of a mid-cerebral artery.

### Route and Dose of ASIC1a Inhibitor Composition Administration

Administration (or administering), as used herein, includes any route of subject exposure to an inhibitor, under any suitable conditions, and at any suitable time(s). Administration may be self-administration or administration by another, such as a health-care practitioner (e.g., a doctor, a nurse, etc.). Administration may be by injection (e.g., intravenous, intramuscular, subcutaneous, intracerebral, epidural, and/or intrathecal, among others), ingestion (e.g., using a capsule, lozenge, a fluid composition, etc.), inhalation (e.g., an aerosol (less than about 10 microns average droplet diameter) inhaled nasally and/or orally), absorption through the skin (e.g., with a skin patch) and/or mucosally (e.g., through oral, nasal, and/or pulmonary mucosa, among others), and/or the like. For administration in connection with brain cancer, intracerebral, intranasal or intrathecal modes may be used. Mucosal administration may be achieved, for example, using a spray (such as a nasal spray), an aerosol that is inhaled), and/or the like. A spray may be a surface spray (droplets on average greater than about 50 microns in diameter) and/or a space spray (droplets on average about 10-50 microns in diameter). In some examples, brain cancer may produce an alteration of the blood-brain barrier of an tumorigenic subject, thus increasing the efficiency with which an inhibitor that is introduced (e.g., by injection and/or absorption) into the bloodstream of a subject can reach the brain. Administration may be performed once or a plurality of times, and at any suitable time relative to brain cancer diagnosis, to provide treatment. Accordingly, administration may be performed before brain cancer has been detected (e.g., prophylactically,) after a minor tumorigenic episode, during chronic brain cancer, after a full tumor, and/or the like.

A therapeutically effective amount of an inhibitor may be administered. A therapeutically effective amount of an inhibitor, as used herein, is any amount of the inhibitor that, when administered to subjects, reduces, in a significant number of the subjects, the degree, incidence, and/or extent of brain cancer-induced injury in the subjects. Accordingly, a therapeutically effective amount may be determined, for example, in clinical studies in which various amounts of the inhibitor are administered to test subjects (and, generally, compared to a control group of subjects).

The inhibitor may be administered in any suitable form and in any suitable composition to subjects. In some examples, the inhibitor may be configured as a pharmaceutically acceptable salt. The composition may be formulated to include, for example, a fluid carrier/solvent (a vehicle), a preservative, one or more excipients, a coloring agent, a flavoring agent, a salt(s), an anti-foaming agent, and/or the like. The inhibitor may be present at a concentration in the vehicle that provides a therapeutically effective amount of the inhibitor for treatment of brain cancer when administered to an tumorigenic subject.

As a general proposition, the therapeutically effective amount of the ASIC1a inhibitor composition administered will be in a weight range of about 1 ng/kg body weight/day to about 100 mg/kg body weight/day whether by one or more administrations. In more particular embodiments, the ASIC1a inhibitor composition is administered in weight range from about 1 ng/kg body weight/day to about 1 μg/kg body weight/day, 1 ng/kg body weight/day to about 100 ng/kg body weight/day, 1 ng/kg body weight/day to about 10 ng/kg body weight/day, 10 ng/kg body weight/day to about 1 μg/kg body weight/day, 10 ng/kg body weight/day to about 100 ng/kg body weight/day, 100 ng/kg body weight/day to about 1 μg/kg body weight/day, 100 ng/kg body weight/day to about 10 μg/kg body weight/day, 1 μg/kg body weight/day to about 10 μg/kg body weight/day, 1 μg/kg body weight/day to about 100 μg/kg body weight/day, 10 μg/kg body weight/day to about 100 μg/kg body weight/day, 10 μg/kg body weight/day to about 1 mg/kg body weight/day, 100 μg/kg body weight/day to about 10 mg/kg body weight/day, 1 mg/kg body weight/day to about 100 mg/kg body weight/day and 10 mg/kg body weight/day to about 100 mg/kg body weight/day.

In other embodiments, the ASIC1a inhibitor composition is administered at a dosage range of 1 ng-10 ng per injection, 10 ng-100 ng per injection, 100 ng-1 μg per injection, 1 μg-10 μg per injection, 10 μg-100 μg per injection, 100 μg-1 mg per injection, 1 mg-10 mg per injection, 10 mg-100 mg per injection, and 100 mg-1000 mg per injection. The ASIC1a inhibitor composition may be injected once daily, twice daily, three times daily, and/or every 2, 3, 4, 5, 6 or 7 days. In addition, the ASIC1a inhibitor composition may be administered over a period of one month, two months, six months, 12 months, 2 years, 5 years, 10 years, 20 years, or more.

In other embodiments, the ASIC1a inhibitor composition may be administered in a range from about 1 ng/kg to about 100 mg/kg. In more particular embodiments, the ASIC1a inhibitor composition may be administered in a range from about 1 ng/kg to about 10 ng/kg, about 10 ng/kg to about 100 ng/kg, about 100 ng/kg to about 1 μg/kg, about 1 μg/kg to about 10 μg/kg, about 10 μg/kg to about 100 μg/kg, about 100 μg/kg to about 1 mg/kg, about 1 mg/kg to about 10 mg/kg, about 10 mg/kg to about 100 mg/kg, about 0.5 mg/kg to about 30 mg/kg, and about 1 mg/kg to about 15 mg/kg.

In other particular embodiments, the amount of the ASIC1a inhibitor composition administered is, or is about, 0.0006, 0.001, 0.003, 0.006, 0.01, 0.03, 0.06, 0.1, 0.3, 0.6, 1, 3, 6, 10, 30, 60, 100, 300, 600 and 1000 mg/day.

The specific dose of the ASIC1a inhibitor composition may be determined based on the particular circumstances of the individual patient including the size, weight, age and sex of the patient, the nature and stage of the disease, the aggressiveness of the disease, and the route of administration of the ASIC1a inhibitor composition.

In certain embodiments, the ASIC1a inhibitor composition may be administered at least once per day, typically once, twice, three times or four times per day with the doses given at equal intervals throughout the day and night to maintain a constant presence of the ASIC1a inhibitor compound to provide sufficient antimicrobial activity. However, a skilled artisan will appreciate that a treatment schedule can be optimized for any given patient, and that administration of compound may occur less frequently than once per day.

In other embodiments, ASIC1a inhibitor composition of the present application is prescribed to be taken in combination with other antimicrobial and/or the other active agents. When used in such combinations, the ASIC1a inhibitor composition of the present application and other antimicrobial agents may be administered simultaneously, by the same or different routes, or at various times during treatment.

The treatment may be carried out for as long a period as necessary, i.e., until the infection is cleared or no longer a threat to the host. In some cases, the treatment may be continued indefinitely while the disease state persists, although discontinuation might be indicated if the antimicrobial compositions no longer produce a beneficial effect. In one embodiment, the treatment is carried out for 6 months and then discontinued. The treating physician can determine whether to increase, decrease, or interrupt treatment based on a patient's response, including evaluation of immune responses, viral loads etc.

### Compositions for Treatment of Brain Cancer

An aspect of the application is a composition for treatment of brain cancer, comprising: an acid sensing ion channel 1a (ASIC1a) inhibitor disposed in a vehicle at a concentration that provides a therapeutically effective amount of the ASIC1a inhibitor for treatment of brain cancer when administered to an tumorigenic subject.

### Pharmaceutical Compositions

As used herein the language “pharmaceutically acceptable carrier” is intended to include all solvents, solubilizers, fillers, stabilizers, binders, absorbents, bases, buffering agents, lubricants, controlled release, vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except as far as any conventional media or agent is incompatible with the active compound, use thereof in the compositions is contemplated. Supplementary agents can also be incorporated into the compositions. In certain embodiments, the pharmaceutically acceptable carrier comprises serum albumin.

The ASIC1a inhibitor pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose, pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene, glycol, and liquid polyethylene glycol, and the like), and suitable mixtures thereof. The proper fluidity can be maintained, for example, using a coating such as lecithin, by the maintenance of the required particle size in the case of dispersion and using surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating an active agent in the required amount in an appropriate solvent with one or a combination of ingredients enumerated above, as required, followed by filtered sterilization. Dispersions can be prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral therapeutic administration, the active compound may be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished using nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and poly lactic acid. Methods for preparation of such formulations will be apparent to those skilled in the art. The materials can also be obtained commercially from e.g., Alza Corporation and Nova Pharmaceuticals, Inc. Liposomal suspensions (including liposomes targeted to infected cells with monoclonal antibodies to viral antigens) can also be used as pharmaceutically acceptable carriers.

It is especially advantageous to formulate oral or parenteral compositions in dosage unit form for ease of administration and uniformity of dosage. Suitable unit dosage forms include, but are not limited to powders, tablets, pills, capsules, lozenges, suppositories, patches, nasal sprays, injectables, implantable sustained-release formulations, lipid complexes, etc.

A dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the present application is dictated by and directly dependent on the unique characteristics of the active compound and the particular therapeutic effect to be achieved, and the limitations inherent in the art of compounding such an active compound for the treatment of individuals.

Toxicity and therapeutic efficacy of the ASIC1a inhibitor composition of the present application can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. ASIC1a inhibitor compounds exhibiting large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to determine useful doses more accurately in humans. The pharmaceutical compositions can be included in a container, pack, or dispenser together with instructions for administration.

The present application is further illustrated by the following examples that should not be construed as limiting. The contents of all references, patents, and published patent applications cited throughout this application, as well as the Figures and Tables, are incorporated herein by reference.

### Method of Manufacturing a Medicament for Treatment of Brain Cancer

An aspect of the application is a method of manufacturing a medicament for treatment of brain cancer, comprising: obtaining an acid sensing ion channel 1a (ASIC1a) inhibitor; and combining the ASIC1a inhibitor with a vehicle to produce a medicament having a therapeutically effective concentration of the inhibitor for administration to an tumorigenic subject for treatment of brain cancer.

An aspect of the application is a use of an acid sensing ion channel 1a (ASIC1a) inhibitor for the manufacture of a medicament to treat brain cancer.

An aspect of the application is a method of manufacturing a medicament for treating brain cancer, comprising formulating an ASIC1a inhibitor into such a medicament.

The following examples describes selected aspects and embodiments of the present teachings, particularly data describing in vitro and in vivo effects of ASIC inhibition, and exemplary cystine knot peptides for use as inhibitors. These examples are intended for the purposes of illustration and should not be construed to limit the scope of the present teachings.

### EXAMPLES

### Materials and Methods

### Reagents and Antibodies

Polyclonal anti-ASIC1 antibody was used for Western blot. Vybrant® MTT cell proliferation assay kit (V-13154) used for cell viability assay was purchased from Thermo Fisher Scientific (MA, USA). SiRNA targeting the human ASIC1 (Product #: NM_001095, siRNA ID: SASI_Hs01_00167899) and SiRNA negative control (SIC001) were synthesized and purified by Sigma-Aldrich (St. Louis, Mo).

### Cell Culture and Transfection

The human glioblastoma cell lines U87MG and A172 were obtained from the American Type Culture Collection (ATCC, Manassas, VA) and cultured with Dulbecco's modified Eagle's medium (DMEM, Sigma-Aldrich, St. Louis, MO) supplemented with 10% fetal bovine serum (FBS), 50 units/ml penicillin and 50 μg/ml streptomycin (PS) at 37° C. in humidified atmosphere incubator containing 5% CO2. SiRNAs (targeting the human ASIC1 and negative control) were transfected using Lipofectamine® RNAiMAX reagent (Invitrogen, Carlsbad, CA) in serum free DMEM medium at a final concentration of 10 nM according to the manufacturer's instructions. After 24 hours of transfection, cells were further grown for 24 hours in growth medium before Western blotting, wound-healing and transwell migration assays.

### Western Blotting

Cells were lysed using M-PER Mammalian Protein Extraction Reagent with protease and phosphatase inhibitor cocktail (Thermo Fisher Scientific, Rockford, USA). Samples were then centrifuged at 13200 g for 20 minutes at 4° C., protein concentrations were measured using Bradford reagent (Bio-Rad Laboratories, Hercules, CA, USA). The supernatant of lysates was transferred to a new tube with 4× loading buff-er and boiled for 10 minutes. All the samples were separated by 10% SDS-PAGE gel, and then transferred onto PVDF membranes (Milli-pore, Germany). Primary antibodies were diluted at the following ratios: rabbit anti-ASIC1 (1:1000), and rabbit anti-β-actin (1:8000). Blots were incubated with primary antibodies overnight at 4° C. followed by the HRP-con-jugated secondary antibodies (Thermo Fisher Scientific, 1:5000) for 1 hour. The signals were identified using an ECL kit (Millipore, Germany) and the band images were acquired using Image Quant LAS 4000.

### Whole-Cell Patch-Clamp Recording

Whole-cell currents were recorded by patch-clamp techniques. The currents were recorded with an Axopatch 200B amplifier and low-pass filtered at 2 KHz, digitized using Digidata 1320 DAC unit. Cells were clamped at a holding potential of −80 mV. The patch pipettes were made from thin walled borosilicate glass micropipettes (1.5 mm diameter, World Precision Instruments, Sarasota, FL) by a micropipette puller (PP83; Tokyo, Japan). The range of pipette resistance was 3-5 MΩ when filled with intracellular solu-tion contained 140 mM CsF, 1 mM CaCl2, 2 mM MgCl2, 10 mM HEPES, 11 mM EGTA, 2 mM tet-raethylammonium chloride, and 4 mM MgATP; pH 7.3 adjusted with CsOH, 290 to 300 mOsm adjusted with sucrose. Cells were perfused with an extracellular fluid (ECF) contained 140 mM NaCl, 5.4 mM KCl, 2 mM CaCl2, 1 mM MgCl2, 10 mM Glucose, and 10 mM HEPES; pH 7.4 and 6.0 adjusted with NaOH, 320-330 mOsm. PcTX1 (Peptide International) was dissolved in ddH2O at 20 μM stock solution before adding to ECF. The data were recorded and analyzed with pClamp and Sigma Plot software.

### Cell Viability Assay

Cells were seeded in 96-well plates overnight and then incubated with pH 7.4 or 7.0 ECF in the absence or presence of 20 nM PcTX1 for 3 h, and then changed to culture medium containing 5% FBS incubating for 24 h, 48 h and 72 h. After the treatments, cell viability was measured by MTT Cell Proliferation Assay Kit according to the manufacturer's instructions. The absorbance values at 540 nm were detected by spectrometer (Molecular devices).

### Wound-Healing Assay

Wound-healing assay was performed. Cells were seeded into a 24-well plate and grown to ˜80% confluence. The cell monolayer was scratched with 200 μl sterile pipette tip and treated with ECF with or without PcTX1 for 3 hours. The original wound areas at 0 hour were photographed with microscope (Olympus FSX100, 4×). After 3 hours, cells were then incubated with culture medium containing 1% FBS for 24 hours. 24 hours later, images were taken from the same location of the original wound areas and the migration distance was measured with Image J software. The wound-healing rate was calculated as: the ratio of wound closure=[(Area of original wound at 0 h−Area of wound after healing at 24 h)/Area of original wound at 0 h]×100%.

### Transwell Migration Assay

The assay is based on two compartments separated by a mem-brane with an accurately defined pore size. Cell transwell chambers with 8-μm pore size of poly-carbonate membrane filters (Corning, USA) were first pre-coated with poly-L-ornithine over-night. Before plating cells, chambers were incubated with serum-free culture medium for 30 minutes. After that, cells were seeded with culture medium containing 1% FBS in the upper-chambers that were immersed to 750 μl whole medium (DMEM with 10% FBS). After incubating for another 24 hours, some cells on the upper surface of the filter migrated through the membrane to the lower surface of the filter. Cells on the upper face of the filter were wiped out thoroughly with cotton swabs, and those attached on the lower surface were fixed with 4% paraformaldehyde for 15 min. They were then stained with 0.5% crystal violet for 10 min, and let the membrane air dry. The cells on the lower surface of the membrane filter were counted under a microscope in 3-4 random fields with 10× magnification. Cell numbers in each group were normalized to the control groups. Assays were performed in at least 3 independent experiments.

### Statistical Analysis

The statistical significance was analyzed using the Prism 8.0.1 software (GraphPad Software, San Diego, CA, USA). The data were expressed as mean±SEM. Groups were compared using one-way ANOVA followed by Tukey's multiple comparison test or unpaired Student's t test where appropriate. P<0.05 was regarded as statistically significant.

### Example 1

### Functional ASIC1 is Expressed in Human Glioma Cells

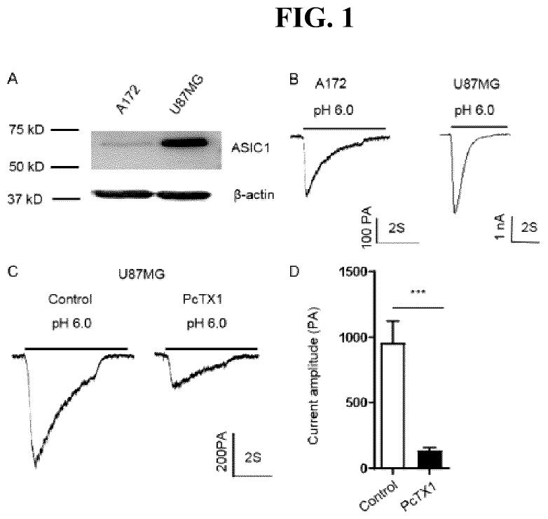

To determine whether ASIC1 is expressed in human glioma cells, we first investigated the expression of ASIC1 protein in A172 and U87-MG cells by Western blotting. Results showed that both of the two glioblastoma cell lines express ASIC1 protein, especially the U87MG cells (FIG. 1A). Next, the study detected the existence of acid-activated currents and the effect of pharmacologic agents on the currents. Acid-activated currents were recorded by whole cell patch-clamp. As shown in FIG. 1B, drop of extra-cellular pH from 7.4 to 6.0 can induce transient inward currents in A172 and U87MG cells. PcTX1 from venom of tarantula Psalmopoeus cambridgei is a specific blocker for homomeric ASIC1a and heteromeric ASIC1a/ASIC2b channels, which potently inhibits the proton-gated currents mediated by ASIC1a. For this reason, the study confirmed the effect of PcTX1 on acid-activated currents. As shown in FIGS. 1C and 1D, the peak of acid-activated currents was decreased by 80% after several minutes of PcTX1 (20 nM) perfusion (***P<0.001, unpaired student's t test, n=5) indicating that the majority of currents activated by extracellular acid are mediated by ASIC1.

### Weak Acid has No Effect on Proliferation of Human Glioma Cells

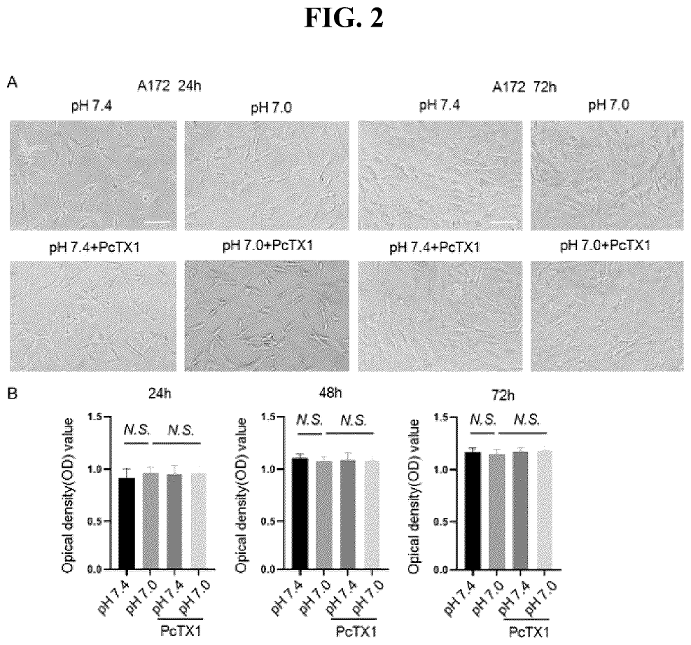

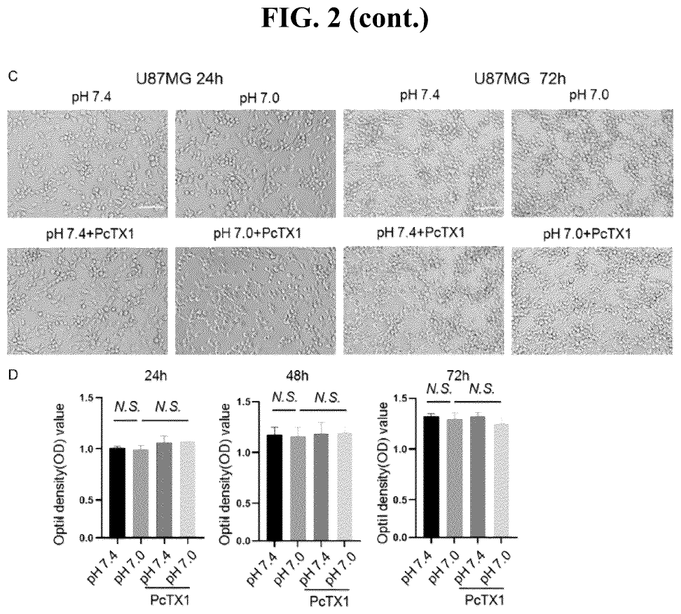

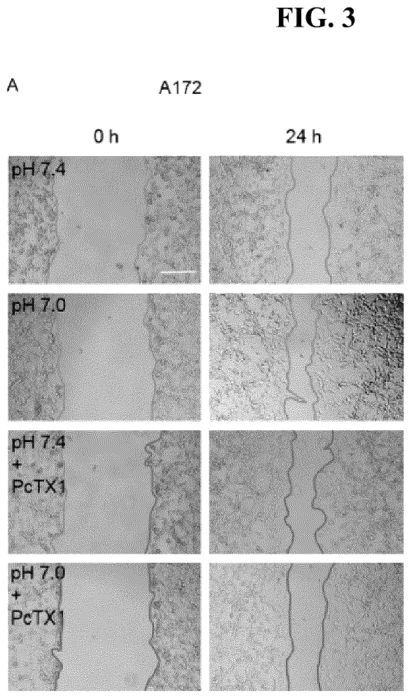

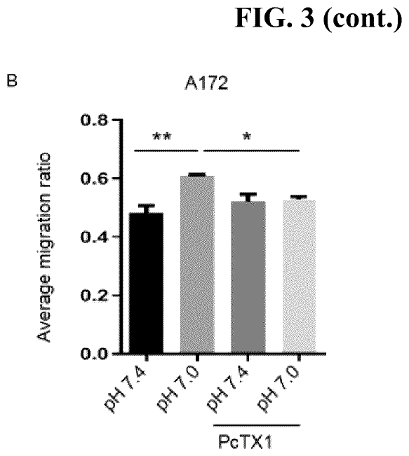

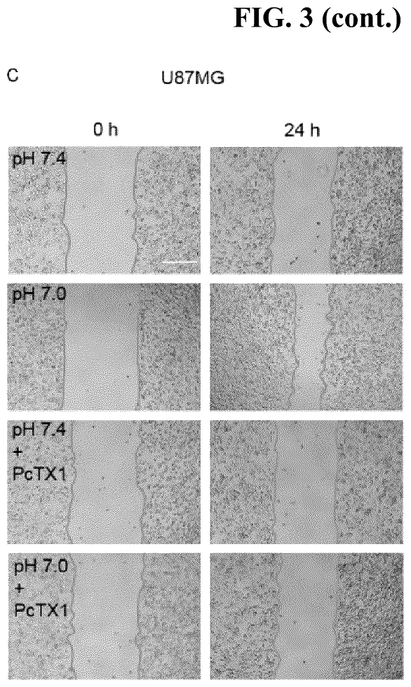

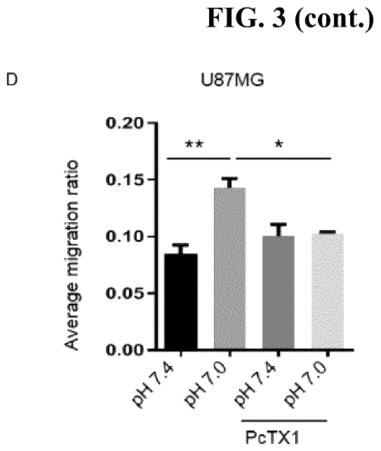

Severe acidosis (e.g. pH 6.0) promotes the proliferation of some type of glioma cells. This study first tested the effect of weak acid on the proliferation of A172 and U87MG cells. The study used either pH 7.4 or pH 7.0 solution to pretreat cells for 3 h, and then changed back to medium containing 5% FBS. The cell viability was measured at 24 h, 48 h and 72 h after acid treatment by MTT assay. Results showed that treatment of cells with pH 7.0 solution had no significant effect on the proliferation of A172 cells at 24 h, 48 h and 72 h, and PcTX1 had no influence on the proliferation of A172 cells (one-way ANOVA, n=3, FIGS. 2A and 2B). Similarly, treatment with weak acid (pH 7.0) and PcTX1 had no clear effect on the proliferation of U87MG cells at different time points (one-way ANOVA, n=3, FIGS. 2C and 2D).

### Weak Acid Promotes the Migration of Human Glioma Cells in Transwell Assay

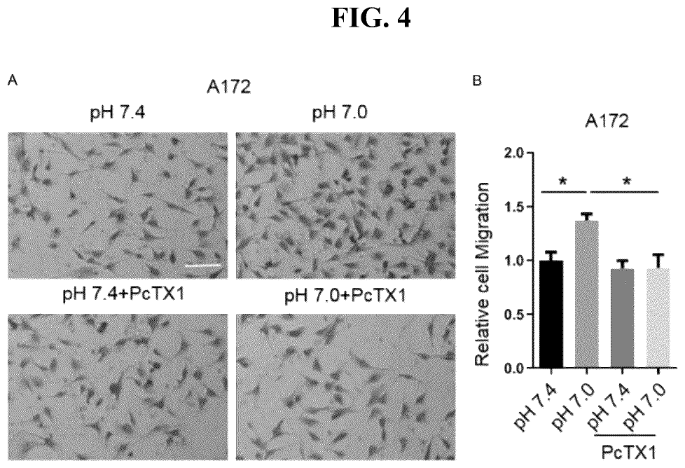

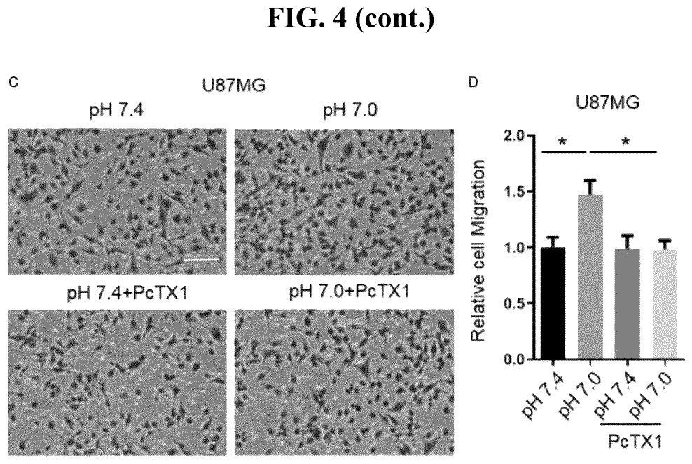

To further examine the role of ASIC1 in glioma cell migration, the study performed a transwell migration assay using inserts with an 8-μm pore size. The glioma cells were cultured in the upper chambers for 24 h, and then pretreated with pH 7.0 in the absence or presence of PcTX1 for 3 h. After 24 h, the number of migrated cells in the lower chamber was examined. Compared with pH 7.4, weak acid (pH 7.0) greatly increased the number of migrated A172 cells by 37%, and PcTX1 (20 nM) completely inhibited the acid induced migration of A172 cells (*P<0.05, one-way ANOVA, n=5, FIGS. 4A and 4B). Similarly, in U87MG cells, weak acid (pH 7.0) increased the migration capacity by 47%. As anticipated, PcTX1 completely blocked the acid-induced increase of migrated U87MG cells (*P<0.05, one-way ANOVA, n=4, FIGS. 4C and 4D).

### Knockdown of ASIC1 Inhibits Migration of Glioma Cells

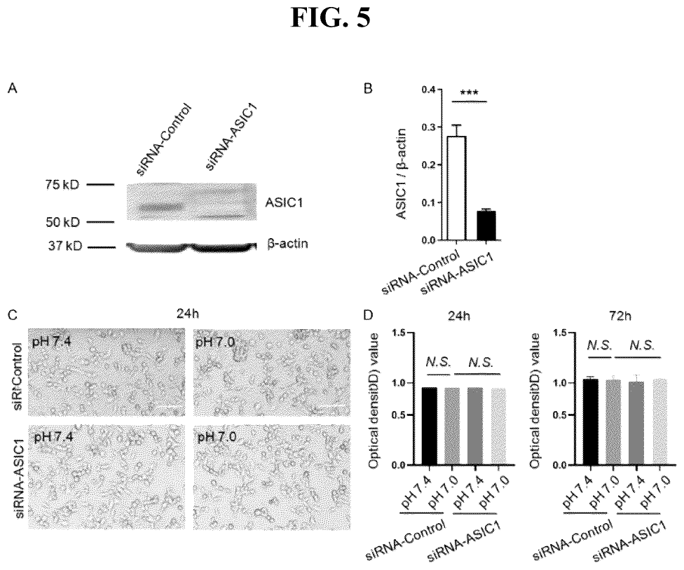

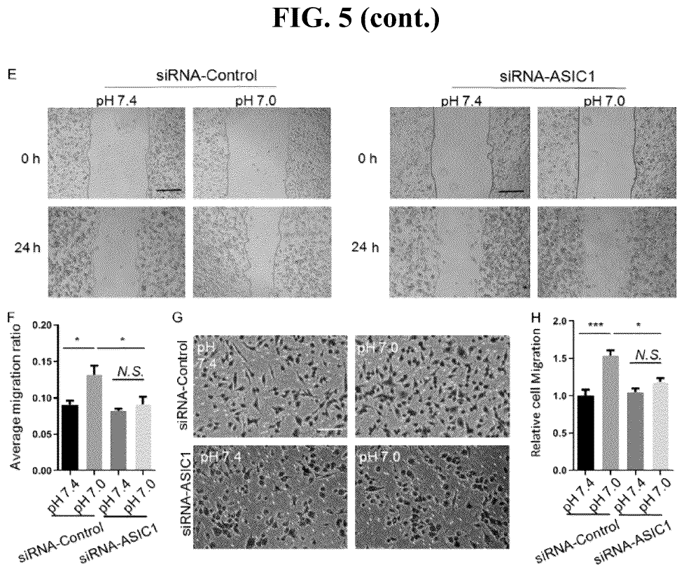

Next, the study used small interference RNA to silence ASIC1 expression and further determined the influence of ASIC1 protein on the migration of glioma cells under acidic condition. Owing to the low ASIC1 expression in A172 cells, the study used U87MG in this experiment to test the effect of ASIC1 siRNA. As shown in FIG. 5A, siRNA-ASIC1 decreased the expression of ASIC1 protein expression in U87MG cells by 72% when compared with siRNA-Control (***P<0.001, unpaired student's t test, n=4, FIGS. 5A and 5B). In certain embodiments, RNA therapies targeting ASIC1 may be used.

Next, the study examined whether knock-down of ASIC1 affects cell proliferation in the presence or absence of extracellular weak acid. MTT assay was performed to detect the cell viability at 24 h and 72 h with and without acid treatment both in siRNA-Control and siRNA-ASIC1 groups. Results showed that knockdown of ASIC1 had no obvious effect on the proliferation of U87MG cells both at 24 h and 72 h either without or with the treatment of extracellular weak acid (one-way ANOVA, n=3, FIGS. 5C and 5D). The study further examined whether knockdown of ASIC1 has an effect on the migration of U87MG cells. As shown in wound healing assay, ASIC1 knockdown did not have an effect on the migration under normal pH (7.4), but it reduced the migration by 44% under acidic condition (pH 7.0) when compared with siRNA-Control (*P<0.05, one-way ANOVA, n=5, FIGS. 5E and 5F). The effect of ASIC1 knockdown on migration was also assessed by transwell. As shown in FIGS. 5G and 5H, weak acid (pH 7.0) produced a large increase in the migration of siRNA-Control treated U87MG cells, however, it had no effect on the migration of siRNA-ASIC1 treated cells (*P<0.05, ***P<0.001, one-way ANOVA, n=4). These results further suggested that ASIC1 is involved in weak acid-induced migration of malignant human glioma cells.

The specific ion transport system is closely related to the characteristics of malignant gliomas, such as significant growth and migration ability. The study then explored the effect of ASIC1 activation on glioma proliferation and migration. As shown in the results, weak acid (pH 7.0) had no effect on the proliferation of A172 and U87MG cells measured both at 24 h and 72 h, but it apparently increased their capacity of migration as shown by wound-healing and transwell assays. Application of ASIC1 inhibitor, PcTX1, effectively blocked the acid-induced migration of the two cells, suggesting that weak acid stimulated glioma migration is mediated, at least in part, by ASIC1 activation. To further determine the role of ASIC1 in the migration of glioma cells, the study also examined the effect of knocking down ASIC1 on glioma migration. The study found that specific siRNA-ASIC1 down-regulated the expression of ASIC1 in U87MG cells and knocking down the ASIC1 channel impaired the migration ability of these cells.

In the present study, it has been shown that the activation of ASIC1 mediates extracellular weak acid induced migration of malignant human glioma cells.

In summary, the study displayed the first evidence that supports a significant role of this channel in the pathogenesis of human gliomas.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended unless the context specifically indicates the contrary.

What is claimed is:

1. A method of treating brain cancer, comprising:
administering a therapeutically effective amount of an acid sensing ion channel 1a (ASIC1a) inhibitor to a tumorigenic subject in order to reduce injury resulting from brain cancer.

2. The method of claim 1, wherein the ASIC1a inhibitor is an amiloride derivative.

3. The method of claim 1, wherein the step of administering includes a step of administering an inhibitor that is selective for ASIC1 a relative to every other member of the ASIC family.

4. The method of claim 1, wherein the step of administering includes a step of administering a peptide that includes a cystine knot.

5. The method of claim 1, wherein the step of administering includes a step of administering a peptide of PcTx1 (SEQ ID NO:1).

6. The method of claim 5, wherein the peptide is a derivative that differs from PcTx1 by at least one deletion, substitution, and/or addition of one or more amino acids.

7. The method of claim 1, the ASIC1a inhibitor being a first inhibitor, further comprising a step of administering a second inhibitor to the subject, the second inhibitor being configured to selectively inhibit at least one other channel that is not a member of the acid sensing ion channel family.

8. The method of claim 1, wherein the step of administering includes a step of administering an inhibitor configured to inhibit ASIC1a channel activity at a concentration of inhibitor that is at least about ten-fold less than for inhibition of another ASIC family member.

9. The method of claim 5, wherein the peptide has an amino acid sequence with at least about 95% identity to PcTx1 (SEQ ID: 1).

10. A method of identifying drugs for treating brain cancer, comprising:
(1) obtaining one or more acid sensing ion channel 1a (ASIC1a) inhibitors; and
(2) testing the one or more ASIC1a inhibitors for an effect, if any, on an tumorigenic subject.

11. The method of claim 10, wherein the step of testing includes a step of (1) selecting a plurality of human subjects that have a tumor, and (2) administering the one or more ASIC1a inhibitors to the human subjects, (3) measuring an effect, of the one or more ASIC1a inhibitors on the subject.

12. A composition for treatment of brain cancer, comprising:
an acid sensing ion channel 1a (ASIC1a) inhibitor disposed in a vehicle at a concentration that provides a therapeutically effective amount of the ASIC1a inhibitor for treatment of brain cancer when administered to a tumorigenic subject.

13. The composition of claim 12, wherein the ASIC1a inhibitor inhibits acid ASIC1a selectively relative to one or more other acid sensing ion channel (ASIC) family members.

14. The composition of claim 12, wherein the ASIC1a inhibitor is a peptide including a cystine knot.

15. The composition of claim 12, wherein the inhibitor is PcTx1 (SEQ ID NO:1).

16. The composition of claim 12, wherein the inhibitor is a derivative of PcTx1.

17. A method of manufacturing the composition of claim 12, comprising:
obtaining the ASIC1a inhibitor; and
combining a therapeutically effective amount of the ASIC1a inhibitor with the vehicle to produce the composition.

18. The method of claim 17, wherein the ASIC1a inhibitor inhibits ASIC1a selectively relative to one or more other acid sensing ion channel (ASIC) family members.

19. The method of claim 17, wherein the ASIC1a inhibitor is PcTx1 (SEQ ID NO: 1) or a derivative of PcTx1.

20. The method of claim 17, wherein the ASIC1a inhibitor inhibits ASIC1a relative to every other member of the ASIC family.
